(19) United States
(12) Patent Application Publication
(10) Pub. No.: US 2024/0149301 A1
(43) Pub. Date: May 9, 2024

(54) ULTRASONIC ARRAY AND ULTRASONIC ARRAY MANUFACTURING METHOD

(71) Applicant: QISDA CORPORATION, Taoyuan City (TW)

(72) Inventor: Fu-Sheng Jiang, Taoyuan City (TW)

(73) Assignee: QISDA CORPORATION, Taoyuan City (TW)

(21) Appl. No.: 18/208,866

(22) Filed: Jun. 12, 2023

(30) Foreign Application Priority Data
Nov. 4, 2022 (TW) 111142118

(51) Int. Cl. B06B 1/06

(52) U.S. Cl. CPC B06B 1/0611

(57) ABSTRACT

An ultrasonic array manufacturing method is applied to an ultrasonic array and includes providing a piezoelectric material layer has at least one or a plurality of piezoelectric units with a first lateral side and a second lateral side opposite to each other, forming a first cutting slot in an oblique manner along a direction from the first lateral side to the second lateral side of each piezoelectric unit, forming a second cutting slot crossed by the first cutting slot on each piezoelectric unit, and coupling two electrode surfaces of each piezoelectric unit respectively to a first lead wire and a second lead wire.

### BACKGROUND OF THE INVENTION

### 1. Field of the Invention

The present invention relates to an ultrasonic array and an ultrasonic array manufacturing method, and more particularly, to an ultrasonic array with preferred resolution and a related ultrasonic array manufacturing method.

### 2. Description of the Prior Art

A conventional ultrasonic detector has a probe that emits ultrasonic signals, and utilizes several piezoelectric devices to emit the ultrasonic signals. Each ultrasonic signal corresponds to a scanning line, and an ultrasonic reflection signal corresponding to the scanning line is received and analyzed to execute an image identification function and an object detection function for medical applications. The piezoelectric material of the conventional ultrasonic detector is divided into several units in a rectangular shape. However, the units arranged in the rectangular shape are difficult to isolate vibration transmission along the structurally longitudinal direction and the diagonal direction of the piezoelectric material, which easily results in noise and affects detection accuracy of the conventional ultrasonic detector.

### SUMMARY OF THE INVENTION

The present invention provides an ultrasonic array with preferred resolution and a related ultrasonic array manufacturing method for solving above drawbacks.

According to the claimed invention, an ultrasonic array manufacturing method includes providing a piezoelectric material layer having at least one or a plurality of piezoelectric units, each of the plurality of piezoelectric units having a first lateral side and a second lateral side corresponding to each other, forming a first cutting slot on each of the plurality of piezoelectric units in an oblique manner along a direction from the first lateral side to the second lateral side, forming a second cutting slot crossed by the first cutting slot on each of the plurality of piezoelectric units in the oblique manner, and forming a second cutting slot crossed by the first cutting slot on each of the plurality of piezoelectric units in the oblique manner; and

According to the claimed invention, an ultrasonic array includes a piezoelectric material layer, a first lead wire and a second lead wire. The piezoelectric material layer has at least one or a plurality of piezoelectric units. Each of the plurality of piezoelectric units has a first lateral side and a second lateral side corresponding to each other, and includes a first cutting slot and a second cutting slot. The first cutting slot is formed on each of the plurality of piezoelectric units in an oblique manner and stretched from the first lateral side to the second lateral side. The second cutting slot is formed on each of the plurality of piezoelectric units in the oblique manner and crossed by the first cutting slot. The first lead wire is coupled to one electrode surface of each of the plurality of piezoelectric units. The second lead wire is coupled to the other electrode surface of each of the plurality of piezoelectric units.

The ultrasonic array provided by the present invention can utilize the ultrasonic array manufacturing method to divide each piezoelectric unit of the piezoelectric material layer into several oblique units. Each oblique unit can be divided by the cutting slots that are not perpendicular to the two corresponding lateral sides of the piezoelectric unit. The oblique units can be staggered to each other, and the cutting slot can be located between the adjacent oblique units, so as to isolate the transmission of vibration along the structurally longitudinal direction and the diagonal directions of the piezoelectric unit. Thus, the ultrasonic array manufacturing method and the related ultrasonic array can have preferred operation bandwidth and be widely applied for the ultrasonic transducer.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is an architecture diagram of an ultrasonic system according to a first embodiment of the present invention.

FIG. 2 is an enlarged diagram of an ultrasonic array according to the first embodiment of the present invention.

FIG. 3 is a diagram of a piezoelectric unit according to the first embodiment of the present invention.

FIG. 4 is an enlarged diagram of the ultrasonic array according to a second embodiment of the present invention.

FIG. 5 is an enlarged diagram of the ultrasonic array according to a third embodiment of the present invention.

FIG. 6 is an enlarged diagram of the ultrasonic array according to a fourth embodiment of the present invention.

FIG. 7 is a flow chart of an ultrasonic array manufacturing method according to an embodiment of the present invention.

FIG. 8 and FIG. 9 are diagrams of the piezoelectric unit according to other embodiments of the present invention.

### DETAILED DESCRIPTION

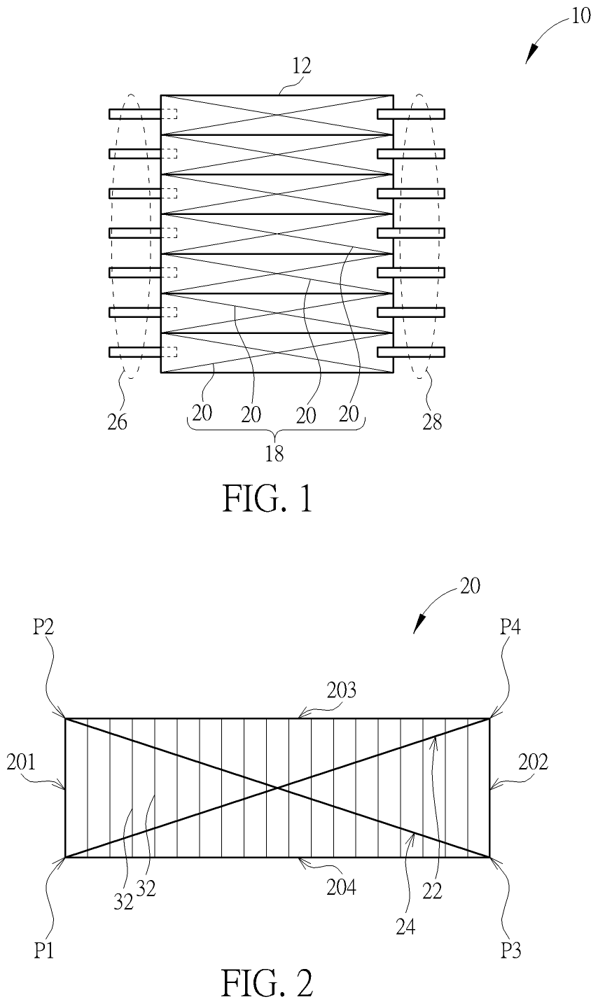

Please refer to FIG. 1. FIG. 1 is an architecture diagram of an ultrasonic system 10 according to a first embodiment of the present invention. The ultrasonic system 10 can include an ultrasonic array 12, and the ultrasonic array 12 can be divided into several units in an oblique manner. The division in the oblique manner can isolate transmission of vibration in multiple directions to improve the resolution of the ultrasonic system 10.

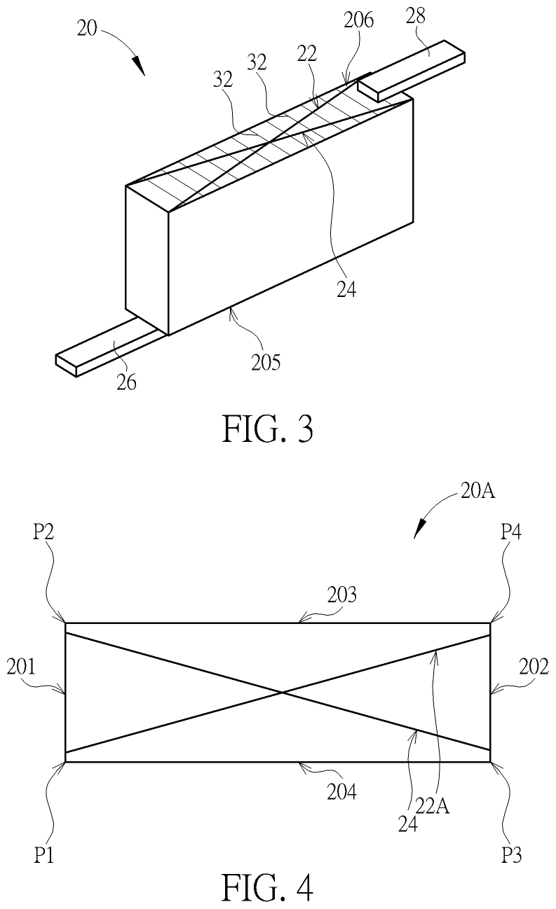

Please refer to FIG. 1 to FIG. 3. FIG. 2 is an enlarged diagram of the ultrasonic array 12 according to the first embodiment of the present invention. FIG. 3 is a diagram of a piezoelectric unit 20 according to the first embodiment of the present invention. A piezoelectric material layer 18 of the ultrasonic array 12 can include a plurality of piezoelectric units 20. Each piezoelectric unit 20 can have a first lateral side 201 and a second lateral side 202 corresponding to each other, and a third lateral side 203 and a fourth lateral side 204 disposed by the first lateral side 201 and the second lateral side 202 and corresponding to each other. The first lateral side 201 and the second lateral side 202 can be short sides of the piezoelectric unit 20. The third lateral side 203 and the fourth lateral side 204 can be long sides of the piezoelectric unit 20. Each piezoelectric unit 20 of the piezoelectric material layer 18 can at least include a first cutting slot 22 and a second cutting slot 24. The first cutting slot 22 can be formed on the piezoelectric unit 20 in the oblique manner from the first lateral side 201 to the second lateral side 202. The second cutting slot 24 can be formed on the piezoelectric unit 20 in the oblique manner and crossed by the first cutting slot 22. An included angle of the second cutting slot 24 relative to the first cutting slot 22 is not limited to the embodiment shown in the figures, and depends on a design demand.

The ultrasonic array 12 can further include a first lead wire 26 and a second lead wire 28. A number of the first lead wire 26 and the second lead wire 28 can correspond to a number of the piezoelectric unit 20 included by the piezoelectric material layer 18. The first lead wire 26 and the second lead wire 28 can be a flexible printed circuit board, or any electronic material with conductive property. Each set of the first lead wire 26 and the second lead wire 28 can be respectively coupled to a lower electrode surface 205 and an upper electrode surface 206 of each piezoelectric unit 20, and can be further stretched outwardly from the first lateral side 201 and the second lateral side 202. The piezoelectric unit 20 set between the first lead wire 26 and the second lead wire 28 can be divided into several sections or units via the first cutting slot 22 and the second cutting slot 24, so as to isolate the transmission of vibration in the multiple directions for increasing the detection efficiency of the ultrasonic system 10.

As shown in FIG. 2, the first lateral side 201 can include a first end point P1 and a second end point P2 corresponding to each other, and the second lateral side 202 can include a third end point P3 and a fourth end point P4 corresponding to each other. The first end point P1 and the third end point P3 can be located on the same side of the piezoelectric unit 20. The second end point P2 and the fourth end point P4 can be located on the other side of the piezoelectric unit 20. In the first embodiment, the first cutting slot 22 can be stretched from the first end point P1 to the fourth end point P4, and the second cutting slot 24 can be stretched from the second end point P2 to the third end point P3. Besides, depths of the first cutting slot 22 and the second cutting slot 24 can be preferably greater than a thickness of the piezoelectric unit 20, or can be greater than or equal to two-thirds of the thickness of the piezoelectric unit 20, which depends on an actual demand.

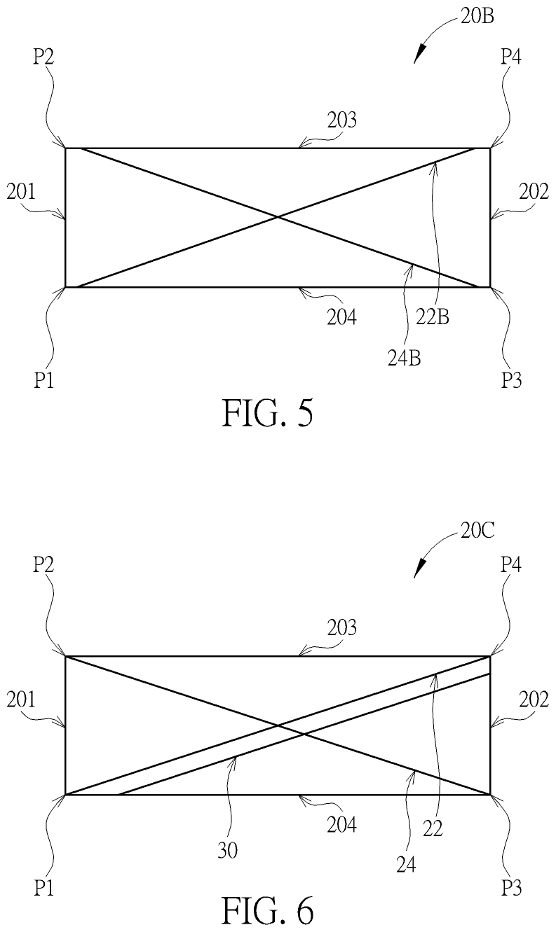

Please refer to FIG. 4 and FIG. 5. FIG. 4 is an enlarged diagram of the ultrasonic array 12A according to a second embodiment of the present invention. FIG. 5 is an enlarged diagram of the ultrasonic array 12B according to a third embodiment of the present invention. In the second embodiment and the third embodiment, elements having the same numerals as ones of the first embodiment have the same structures and functions, and a detailed description is omitted herein for simplicity. As shown in FIG. 4, the first cutting slot 22A of the ultrasonic array 12A can be stretched from position adjacent to the first end point P1 to position adjacent to the fourth end point P4, and two opposite ends of the first cutting slot 22A can be respectively staggered to the first lateral side 201 and the second lateral side 202; two opposite ends of the second cutting slot 24A can be respectively staggered to the first lateral side 201 and the second lateral side 202.

As shown in FIG. 5, the first cutting slot 22B of the ultrasonic array 12B can be stretched from the position adjacent to the first end point P1 to the position adjacent to the fourth end point P4, and two opposite ends of the first cutting slot 22B can be respectively staggered to the third lateral side 203 and the fourth lateral side 204; two opposite ends of the second cutting slot 24B can be respectively staggered to the third lateral side 203 and the fourth lateral side 204. In the foresaid embodiments, the first cutting slot 22 (or the first cutting slot 22A or the first cutting slot 22B) can be symmetrical to the second cutting slot 24 (or the second cutting slot 24A or the second cutting slot 24B); however, the first cutting slot 22 and the second cutting slot 24 may be asymmetric. For example, the two opposite ends of the first cutting slot 22 can be respectively staggered to the first lateral side 201 and the third lateral side 203, or the two opposite ends of the second cutting slot 24 can be respectively staggered to the third lateral side 203 and the second lateral side 202. Variation of the first cutting slot 22 and the second cutting slot 24 can depend on the design demand.

Please refer to FIG. 6. FIG. 6 is an enlarged diagram of the ultrasonic array 12C according to a fourth embodiment of the present invention. In this embodiment, elements having the same numerals as ones of the foresaid embodiments have the same structures and functions, and the detailed description is omitted herein for simplicity. The piezoelectric material layer 18 of the ultrasonic array 12C can further include at least one third cutting slot 30 formed on the piezoelectric unit 20 and parallel to or substantially parallel to the first cutting slot 22, or a small included angle can be formed between the third cutting slot 30 and the first cutting slot 22. The fourth embodiment can optionally form another cutting slot (which is not shown in the figures) parallel to or staggered to the second cutting slot 24, and the foresaid cutting slot can have functions similar to ones of the third cutting slot 30. Therefore, the ultrasonic array 12 of the present invention can form several obliquely cutting slots with similar or different depths on each piezoelectric unit 20 of the piezoelectric material layer 18 in accordance with the design demand.

As shown in FIG. 2 and FIG. 3, the piezoelectric material layer 18 of the first embodiment can further include a fourth cutting slot 32 formed on the piezoelectric unit 20 and parallel to the first lateral side 201 or the second lateral side 202. The fourth cutting slot 32 can be further applied for the second embodiment, the third embodiment and the fourth embodiment. The fourth cutting slot 32 can be staggered to the first cutting slot 22 and the second cutting slot 24. A number and a density of the fourth cutting slot 32 cannot be limited to the embodiments shown in FIG. 2 and FIG. 3; generally, a distance between two adjacent fourth cutting slots 32 can be ranged between 0.01 mm˜0.2 mm, and an actual value of the said distance can depend on the design demand.

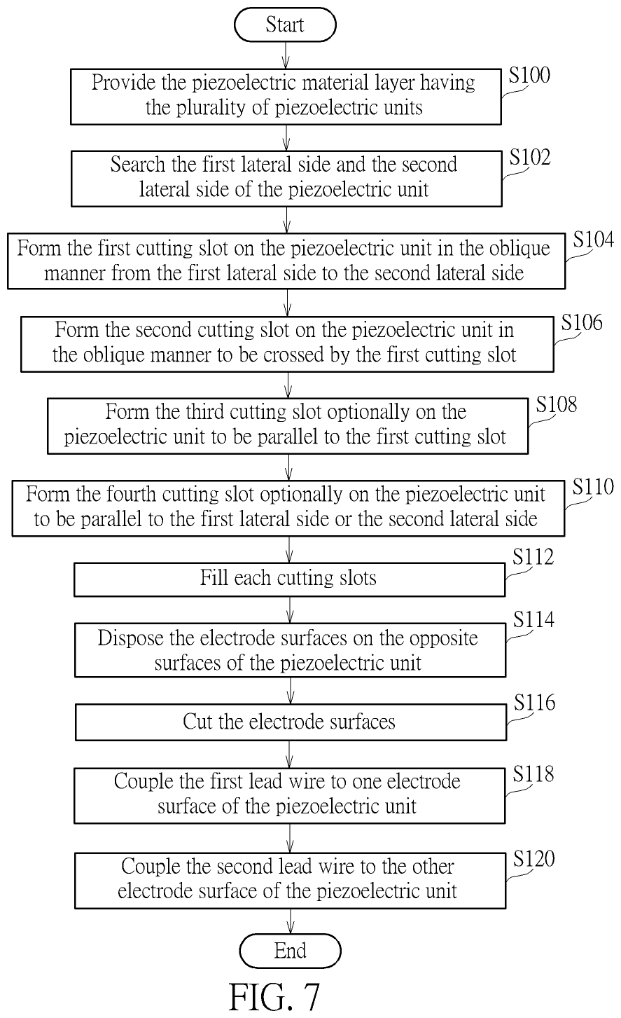

Please refer to FIG. 7. FIG. 7 is a flow chart of an ultrasonic array manufacturing method according to an embodiment of the present invention. The ultrasonic array manufacturing method illustrated in FIG. 7 can be suitable for the ultrasonic system 10 shown in FIG. 1. The ultrasonic array manufacturing method can include steps S100˜S114 as following:

Step S100: provide the piezoelectric material layer 18 having the plurality of piezoelectric units 20;

Step S102: search the four lateral sides of the each piezoelectric unit 20, and define the two short sides of the four lateral sides as the first lateral side 201 and the second lateral side 202 of the piezoelectric unit 20;

Step S104: form the first cutting slot 22 on the piezoelectric unit 20 in the oblique manner from the first lateral side 201 to the second lateral side 202;

Step S106: form the second cutting slot 24 on the piezoelectric unit 20 in the oblique manner to be crossed by the first cutting slot 22;

Step S108: form the third cutting slot 30 on the piezoelectric unit 20 to be parallel to the first cutting slot 22;

Step S110: form the fourth cutting slot 32 on the piezoelectric unit 20 to be parallel to the first lateral side 201 or the second lateral side 202;

Step S112: fill each cutting slots by silicone, epoxy, or equivalent material;

Step S114: dispose the lower electrode surface 205 and the upper electrode surface 206 respectively on the two opposite surfaces of the piezoelectric unit 20;

Step S116: cut the lower electrode surface 205 and the upper electrode surface 206 disposed on the opposite surfaces of the piezoelectric unit 20 along a direction of the fourth cutting slot 32;

Step S118: couple the first lead wire 26 to the lower electrode surface 205 of each piezoelectric unit 20;

Step S120: couple the second lead wire 28 to the upper electrode surface 206 of each piezoelectric unit 20.

The details of step S100 to step S114 can be referred to the above-mentioned description, and can be omitted herein for simplicity. The included angle between the first cutting slot 22 and the second cutting slot 24 formed in step S104 and step S106 can be changed in accordance with different embodiments. Step S108 and step S110 can be optional steps. The ultrasonic array manufacturing method can decide whether to form the third cutting slot 30 and the fourth cutting slot 32, and how to set the included angle and the position of the third cutting slot 30 and the fourth cutting slot 32 in accordance with the design demand. Step S112 to step S120 can fill the first cutting slot 22, the second cutting slot 24, the third cutting slot 30 and the fourth cutting slot 32 by silicone, epoxy, or any equivalent material, and dispose the lower electrode surface 205 and the upper electrode surface 206 on the opposite surfaces of the piezoelectric unit 20 in an electroplating or other manners (or bonding the flexible cable to the adjacent piezoelectric unit 20); then, the lower electrode surface 205 and the upper electrode surface 206 can be cut along the direction of the fourth cutting slot 32, and the first lead wire 26 and the second lead wire 28 can be respectively coupled to the lower electrode surface 205 and the upper electrode surface 206. The ultrasonic array 12 of the ultrasonic system 10 can be manufactured by the ultrasonic array manufacturing method illustrated in steps S100-S120. Each piezoelectric unit 20 of the ultrasonic array 12 can be divided into several oblique units via the plurality of obliquely cutting slots; each oblique unit can provide its detection function in a specific angle, and air or a filler (such as epoxy or silicone or adhesive) within the cutting slot between the adjacent oblique units can isolate the transmission of vibration, so as to increase the object detection resolution of the ultrasonic system 10.

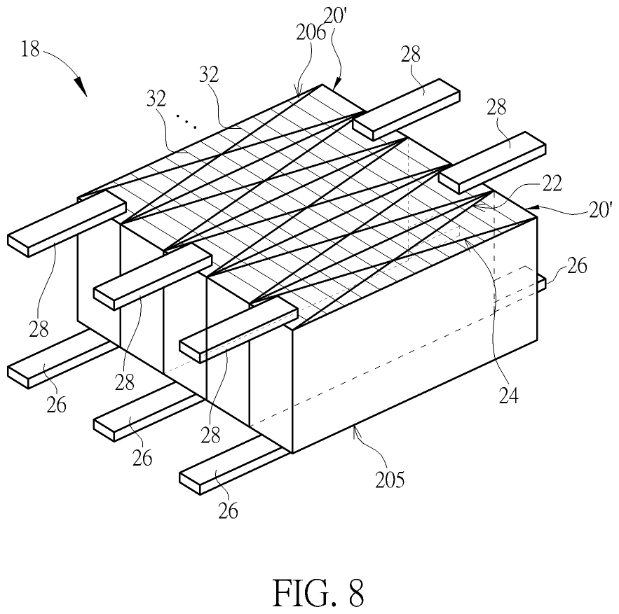

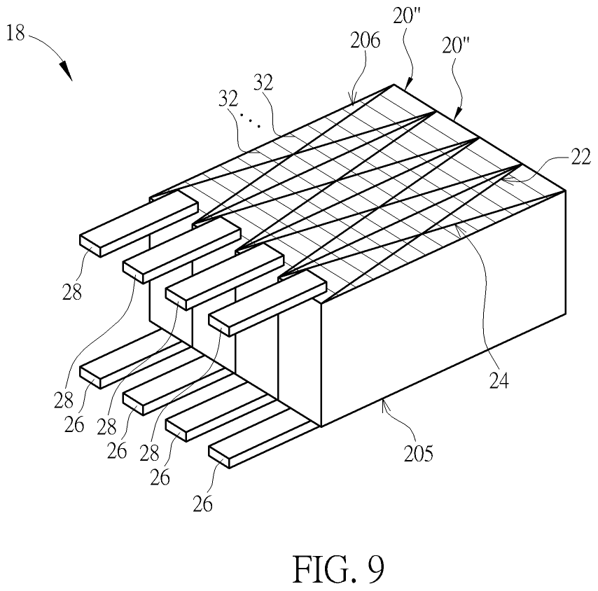

Please refer to FIG. 8 and FIG. 9. FIG. 8 and FIG. 9 are diagrams of the piezoelectric unit 20′ and 20″ according to other embodiments of the present invention. The first lead wire 26 and the second lead wire 28 (which are shown in FIG. 3) can be stretched outwardly from the first lateral side 201 and the second lateral side 202 of the piezoelectric unit 20; however, an actual application is not limited to the foresaid embodiment. As shown in FIG. 8, the first lead wire 26 and the second lead wire 28 of each piezoelectric unit 20′ can be stretched outwardly from the same short side (such as the first lateral side 201 or the second lateral side 202), and the lead wires of the adjacent piezoelectric units 20′ can be staggered and stretched toward different short sides. As shown in FIG. 9, the first lead wire 26 and the second lead wire 28 of each piezoelectric unit 20″ can be stretched outwardly from the same short side (such as the first lateral side 201 or the second lateral side 202), and the lead wires of the adjacent piezoelectric units 20″ can be stretched outwardly from the same short side.

In conclusion, the ultrasonic array provided by the present invention can utilize the ultrasonic array manufacturing method to divide each piezoelectric unit of the piezoelectric material layer into several oblique units. Each oblique unit can be divided by the cutting slots that are not perpendicular to the two corresponding lateral sides of the piezoelectric unit. The oblique units can be staggered to each other, and the cutting slot can be located between the adjacent oblique units, so as to isolate the transmission of vibration along the structurally longitudinal direction and the diagonal directions of the piezoelectric unit. Thus, the ultrasonic array manufacturing method and the related ultrasonic array can have preferred operation bandwidth and be widely applied for the ultrasonic transducer.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An ultrasonic array manufacturing method, comprising:
providing a piezoelectric material layer having at least one or a plurality of piezoelectric units, wherein each of the plurality of piezoelectric units has a first lateral side and a second lateral side corresponding to each other;
forming a first cutting slot on each of the plurality of piezoelectric units in an oblique manner along a direction from the first lateral side to the second lateral side;
forming a second cutting slot crossed by the first cutting slot on each of the plurality of piezoelectric units in the oblique manner; and
coupling two electrode surfaces of each of the plurality of piezoelectric units respectively to a first lead wire and a second lead wire.

2. The ultrasonic array manufacturing method of claim 1, further comprising:
forming a third cutting slot parallel to the first cutting slot on each of the plurality of piezoelectric units.

3. The ultrasonic array manufacturing method of claim 1, further comprising:
searching four lateral sides of each of the plurality of piezoelectric units to define two corresponding short sides of the four lateral sides as the first lateral side and the second lateral side.

4. The ultrasonic array manufacturing method of claim 1, wherein the first lateral side comprises a first end point and a second end point opposite to each other, the second lateral side comprises a third end point and a fourth end point opposite to each other, the first end point and the third end point are located on the same side of each of the plurality of piezoelectric units, the second end point and the fourth end point are located on the other side of each of the plurality of piezoelectric units, the ultrasonic array manufacturing method further comprises:
forming the first cutting slot from position adjacent to the first end point to position adjacent to the fourth end point; and
forming the second cutting slot from position adjacent to the second end point to position adjacent to the third end point.

5. The ultrasonic array manufacturing method of claim 1, wherein two opposite ends of the first cutting slot are respectively staggered to the first lateral side and the second lateral side.

6. The ultrasonic array manufacturing method of claim 1, wherein each of the plurality of piezoelectric units further has a third lateral side and a fourth lateral side located by the first lateral side and the second lateral side and corresponding to each other, and two opposite ends of the first cutting slot are respectively staggered to the third lateral side and the fourth lateral side.

7. The ultrasonic array manufacturing method of claim 1, wherein a depth of the first cutting slot is greater than a thickness of each of the plurality of piezoelectric units, or is greater than or equal to two-thirds of the thickness of each of the plurality of piezoelectric units.

8. The ultrasonic array manufacturing method of claim 1, wherein the first cutting slot is symmetrical to the second cutting slot.

9. The ultrasonic array manufacturing method of claim 1, further comprising:
forming a plurality of fourth cutting slots parallel to the first lateral side or the second lateral side on each of the plurality of piezoelectric units.

10. The ultrasonic array manufacturing method of claim 9, wherein a distance between two adjacent fourth cutting slots of the plurality of fourth cutting slots is ranged between 0.01 mm 0.2 mm.

11. An ultrasonic array, comprising:
a piezoelectric material layer, having at least one or a plurality of piezoelectric units, each of the plurality of piezoelectric units having a first lateral side and a second lateral side corresponding to each other, and comprising:
a first cutting slot formed on each of the plurality of piezoelectric units in an oblique manner and stretched from the first lateral side to the second lateral side; and
a second cutting slot formed on each of the plurality of piezoelectric units in the oblique manner and crossed by the first cutting slot;
a first lead wire coupled to one electrode surface of each of the plurality of piezoelectric units; and
a second lead wire coupled to the other electrode surface of each of the plurality of piezoelectric units.

12. The ultrasonic array of claim 11, wherein the piezoelectric material layer further has a third cutting slot formed on each of the plurality of piezoelectric units and parallel to the first cutting slot.

13. The ultrasonic array of claim 11, wherein two corresponding short sides of four lateral sides on each of the plurality of piezoelectric units are defined as the first lateral side and the second lateral side.

14. The ultrasonic array of claim 11, wherein the first lateral side comprises a first end point and a second end point opposite to each other, the second lateral side comprises a third end point and a fourth end point opposite to each other, the first end point and the third end point are located on the same side of each of the plurality of piezoelectric units, the second end point and the fourth end point are located on the other side of each of the plurality of piezoelectric units, the first cutting slot is formed from position adjacent to the first end point to position adjacent to the fourth end point, the second cutting slot is formed from position adjacent to the second end point to position adjacent to the third end point.

15. The ultrasonic array of claim 11, wherein two opposite ends of the first cutting slot are respectively staggered to the first lateral side and the second lateral side.

16. The ultrasonic array of claim 11, wherein each of the plurality of piezoelectric units further has a third lateral side and a fourth lateral side located by the first lateral side and the second lateral side and corresponding to each other, and two opposite ends of the first cutting slot are respectively staggered to the third lateral side and the fourth lateral side.

17. The ultrasonic array of claim 11, wherein a depth of the first cutting slot is greater than a thickness of each of the plurality of piezoelectric units, or is greater than or equal to two-thirds of the thickness of each of the plurality of piezoelectric units.

18. The ultrasonic array of claim 11, wherein the first cutting slot is symmetrical to the second cutting slot.

19. The ultrasonic array of claim 11, wherein the piezoelectric material layer further comprises a plurality of fourth cutting slots formed on each of the plurality of piezoelectric units and parallel to the first lateral side or the second lateral side.

20. The ultrasonic array of claim 19, wherein a distance between two adjacent fourth cutting slots of the plurality of fourth cutting slots is ranged between 0.01 mm˜0.2 mm.
